(19) United States
(12) Patent Application Publication
(10) Pub. No.: US 2026/0034543 A1
(43) Pub. Date: Feb. 5, 2026

(54) MICROFLUIDIC DEVICE AND MICROFLUIDIC SYSTEM

(71) Applicant: ibidi GmbH, Gräfelfing (DE)

(72) Inventors: Jan SCHWARZ, Bingen (DE); Roman ZANTL, Gräfelfing (DE); Nina HUBER, Dachau (DE); Georg LUBINS, München (DE)

(21) Appl. No.: 19/274,292

(22) Filed: Jul. 18, 2025

(30) Foreign Application Priority Data
Jul. 31, 2024 (EP) 24192130.3

(51) Int. Cl. B01L 3/00; C12M 1/00

(52) U.S. Cl. CPC B01L 3/502715; C12M 41/14; B01L 2200/0642; B01L 2200/16; B01L 2300/0809; B01L 2300/0851; B01L 2400/0478; B01L 2400/0622

(57) ABSTRACT

A microfluidic device includes a substrate, a channel system arranged in the substrate, a first and a second fluidic connection for supplying a fluid into the channel system, the fluidic connections being arranged on a first side of the substrate, a first and a second valve seat, both formed in a second side of the substrate, which is opposite to the first side, and an elastic membrane covering at least a portion of the second side of the substrate including the valve seats, wherein the first valve seat is arranged such that the fluid can flow from the first connection through the first valve seat into the channel system, and wherein the second valve seat is arranged such that the fluid can flow from the second connection through the second valve seat into the channel system. A microfluidic system may include the microfluidic device and an actuator device.

### RELATED APPLICATIONS

This application claims the benefit under 35 U.S.C. § 119(a) of European Patent Application No. 24 192 130.3 filed Jul. 31, 2024, which is incorporated by reference herein.

### TECHNICAL FIELD

The present invention relates to a microfluidic device and a microfluidic system comprising the microfluidic device.

### BACKGROUND

In a variety of cell biology applications, liquids or gases (both are collectively referred to as ‘fluid’) are passed through a microfluidic system to perform studies of biofilms or cell aggregates and to study the behavior of cells under precisely controllable flow conditions. For this purpose, the cells can be present in an observation chamber on a chip or in a substrate, and the substrate is connected to the microfluidic system with which the fluid is passed through the observation chamber. This setup is used, for example, in arteriosclerosis research in connection with the adhesion behavior of cells, or also in the simulation of organ models, whereby physiological conditions such as those in an organ (e.g. the intestine) are imitated with the aid of the microfluidic system. A microfluidic system with such an observation chamber can be placed in an incubator so that the cells can be cultivated in it. It is desirable for microfluidic systems to be highly flexible with regard to the arrangement of different system components. For example, it should be possible to add fluids without changing the existing flow conditions for the samples under investigation.

A microfluidic system is known from EP 1 944 084 A1 in which a liquid is passed between two reservoirs through an observation chamber. Regardless of the pumping direction, the liquid always flows through the observation chamber in the same direction thanks to a rectifier arrangement of valves.

It may be necessary to add further components to the liquid as required, for example nutrient solutions, enzymes, messenger substances, medication, etc., without disturbing the existing flow. However, the existing flow should not be disturbed, as this could have undesirable effects on the cells under investigation. The state of the art does not yet provide a satisfactory solution to this problem.

### BRIEF DESCRIPTION OF THE DRAWINGS

Features and advantages of the invention are explained below with reference to the exemplary figures.

FIG. 1A shows an oblique view of a microfluidic device;

FIG. 1B shows a further oblique view of the microfluidic device according to FIG. 1A;

FIG. 1C shows an exploded view of the microfluidic device according to FIG. 1A;

FIG. 2A shows an oblique view of a microfluidic system;

FIG. 2B shows a block diagram of a microfluidic system as shown in FIG. 2A;

FIG. 3 shows a detailed view of a valve in a microfluidic system;

FIG. 4A shows a schematic circuit diagram of a microfluidic system;

FIG. 4B shows a simplified circuit diagram of the valve arrangement in a microfluidic system according to FIG. 4A

FIG. 4C shows a schematic diagram of the actuator device of the microfluidic system of FIG. 4A;

FIG. 5A shows a schematic circuit diagram of a microfluidic system;

FIG. 5B shows a simplified circuit diagram of the valve arrangement in a microfluidic system according to FIG. 5A;

FIG. 5C shows a schematic diagram of the actuator device of the microfluidic system of FIG. 5A;

FIG. 6 shows an oblique view of a microfluidic system; and

FIG. 7 shows an oblique view of another microfluidic system.

### DETAILED DESCRIPTION

Embodiments of the present invention provide a microfluidic device in which various components can be added to a fluid flow in the microfluidic device without impairing the existing cultivation conditions for cells under a fluid flow in the microfluidic device or a microfluidic system.

This task is solved by the microfluidic device described in greater detail below.

According to embodiments of the invention, there is provided a microfluidic device comprising a substrate, a channel system arranged in the substrate, a first and a second fluidic connection for supplying a fluid into the channel system, the fluidic connections being arranged on a first side of the substrate, a first and a second valve seat both configured in a second side of the substrate opposite to the first side, and an elastic membrane covering at least a part of the second side of the substrate including the valve seats. Here, the first valve seat is arranged such that the fluid can flow from the first connection through the first valve seat into the channel system, and the second valve seat is arranged such that the fluid can flow from the second connection through the second valve seat into the channel system.

Fluid reservoirs can be connected to one or more of the fluidic connections so that the fluid flows from the respective fluid reservoir into the channel system when the valves are open. The targeted opening and closing of the valve seats controls which fluids flow into the channel system and in what quantity. This allows a precise mixing ratio of fluids to be created in the channel system. Furthermore, the channel system can be connected to an observation chamber in which a microbiological sample is located. This sample is supplied with the fluid mixture from the channel system. If the fluids are (liquid) nutrient solutions, for example, the sample can be precisely supplied with nutrients. In this respect, the microfluidic system can provide a precisely coordinated nutrient solution for microbiological samples. Furthermore, if fluids are added to the channel system exclusively through the valve seats, the flow conditions in the channel system of the microfluidic device and a connected observation chamber are not affected.

The fluid can be a liquid and/or a gas.

The substrate may comprise or consist of a plastic. In particular, it may comprise plastics such as COC (cyclo-olefin copolymer), COP (cyclo-olefin polymer), PC (polycarbonate), PS (polystyrene), PE (polyethylene), PMMA (polymethyl methacrylate) or a transparent thermoplastic or an elastomer or consist of one of these plastics or a mixture thereof.

The substrate can be an injection-molded part, i.e. manufactured by injection molding. By using the aforementioned materials and processes, a microfluidic device can be produced cost-effectively and in large quantities with consistent quality.

Alternatively, the substrate can comprise a glass. In particular, the glass or plastic may have the birefringence and autofluorescence of a Schott cover glass (such as D 263 M Schott glass, No. 1.5 H (170+/−10 μm)).

The substrate can be transparent, especially in the visible wavelength range. This allows the associated microfluidic device to be used for optical investigations, for example (inverse) microscopy.

Such an optically high-quality material can enable microscopy examinations with high precision and low optical imperfections.

The substrate and the fluidic connections can be made of the same material. Likewise, the fluidic connections can be part of the substrate. Furthermore, the substrate with the fluidic connections can be manufactured as one piece. This further simplifies the manufacturability of the microfluidic device.

The channel system configured in the substrate connects the fluidic connections of the microfluidic device to each other. The channel system may comprise one channel or a plurality of channels. In the case of a plurality of channels, these can be connected to each other and form a network. A channel can be a cavity configured in the substrate or a trench configured in the substrate, which is covered by another element, for example a film.

The valve seats are arranged in such a way that a fluid must flow from one of the fluidic connections through the valve seat to enter the channel system. It is therefore possible to control if and when the flow to the channel system is released from the connections. If there is already a fluid in the channel system, the addition of further substances can be controlled from the outside without having to interrupt an existing flow in the channel system. This means that the cultivation conditions for cells exposed to the flow in the channel system or in an observation chamber connected to the channel system remain essentially unchanged and are only influenced in the desired way by the substances added.

A valve seat refers to the part of a valve through which the fluid flows in the open state of the associated valve, with the flow being from the fluidic connection into the channel system or in the reverse direction. Accordingly, the flow is blocked in a closed state of the associated valve. The closed state can be achieved by pressing the membrane into the valve seat.

The first and second fluidic connections are arranged on the first side. The first side can be configured to be essentially flat. The connections can protrude from the flat first side or project into the substrate.

The second side of the substrate can be configured to be flat. Alternatively, the second side may have a recess in which the membrane is arranged. The recess can have the same thickness or depth as the membrane, so that the membrane and the second side are configured to be flush. The membrane is therefore flush with the second side. An underside of the microfluidic device, i.e. the second side of the substrate with the applied membrane, can be flat in this case.

The microfluidic device is configured in particular for applications with (inverted) microscopy, in which the substrate or the microfluidic device (with the membrane) acts as a microscope slide. A flat surface reduces optical imperfections and improves the stability of the microfluidic device on a microscope stage.

The channel system can be configured at least partially in the form of a trench on the second side of the substrate. The membrane can seal the trench fluidically. Alternatively, a film, in particular an adhesive film, can cover the trench. The trench can thus be bounded on one side by the membrane or the film and on the other sides by the substrate. The channel system may comprise one or more channels. All channels or individual channels can be configured in the form of a trench.

The structure of a trench in the surface of the substrate can be easily created by injection molding.

The channel system may be connected to the valve seats such that the fluid can flow from the valve seat into the channel system or from the channel system into the valve seat when the valve seat is in the open state. In other words, there may be a fluidic connection between the channel system and the valve seats, whereby this connection is only open for fluid flow when the membrane is not actively pressed into the valve seat.

The valve seat may be an orifice between a channel system and a through hole through the substrate from the first side to the second side. From a connection on the first side of the substrate, the fluid can flow through the through hole to the second side of the substrate where there is a connection to the channel system. This connection can be blocked when the membrane is pressed into or onto the orifice of the through hole. The valve seat is then closed and no fluid enters the channel system from the connection.

The membrane can be a silicone membrane. The silicone membrane can comprise or consist of Elastosil. Similarly, the membrane may comprise or consist of a thermoplastic elastomer such as Flexdym. Alternatively, the membrane may comprise or consist of a COC.

By choosing these materials, the membrane is capable of withstanding a large number of switching cycles without compromising its elasticity or its ability to close the valve.

Furthermore, the membrane may have been manufactured by injection molding. In particular, the membrane can be an elastic silicone injection molded part. This provides both the advantages of injection molding and the advantages of silicone as a material for the membrane.

The membrane may comprise several layers. For example, a first layer can reduce buckling due to elasticity fatigue. A second layer may have high elasticity to improve sealing properties against the valve seat.

The membrane may have a thickness in the range between 5 μm and 5 mm, in particular between 50 μm and 250 μm.

The thickness of the membrane is selected so that the membrane can withstand a high number of switching cycles with constant elasticity. A membrane that is too thin could, for example, tear under pressure or lose its elasticity after only a short period of use. A membrane that is too thick, on the other hand, would require a large force to press it into the valve seat.

The membrane can be transparent, especially in the visible wavelength range. This allows optical examinations, for example with an (inverted) microscope through the membrane.

Furthermore, a double-sided adhesive film can be arranged between the substrate and the membrane, and the membrane can be attached to the substrate by means of the adhesive film. The adhesive film can leave out the valve seats. The membrane can also leave out the trench.

Attaching or fixing the membrane to the substrate by means of an adhesive film is a simple and effective method of attachment.

In this case, the trench in the second side of the substrate may have a thickness or depth corresponding to the sum of the thicknesses of the membrane and the adhesive film. Thus, when the membrane is attached with an adhesive film, a flush finish with the second side of the substrate can be achieved. The underside of the microfluidic device is thus flat.

Alternatively, the membrane can be attached to the substrate by ultrasonic welding or solvent welding, in particular also using claw effects in openings or roughness provided for this purpose. A clamp connection is also conceivable, in which the membrane is clamped to the substrate by means of a sealing lip. Adhesive or the like is not required with this method.

The microfluidic device can also comprise an observation chamber that is fluidically connected to the channel system via an inlet and an outlet. In particular, the observation chamber may be formed in the substrate. The inlet and the outlet can be orifices on the channel system. Alternatively, the inlet and the outlet can be connecting tubes between the channel system and the observation chamber. The inlet and outlet can be separate channels that are not part of the channel system.

The observation chamber can be used to store and cultivate cells, cell aggregates or other biological samples to be examined. The cells are also controlled there under certain environmental conditions, including the supply of nutrients. The observation chamber is connected to the channel system via an inlet and an outlet, so that a liquid from the channel system is also supplied to the observation chamber. In particular, a flow of liquid in the channel system also leads to a flow in the observation chamber. This flow allows said cells to be cultivated in the observation chamber, for example by supplying nutrients via the liquid.

If the observation chamber is formed in the substrate, the substrate may only be transparent in the area of the observation chamber, in particular in the visible wavelength range. In the other areas, however, the substrate can be opaque or translucent. Microscopic observations are generally only carried out in the area of the observation chamber, while the other areas of the substrate are only of minor relevance for such observations. In this respect, the optical quality requirements for the other areas are significantly lower, meaning that more cost-effective materials can be used.

A further valve seat can be arranged between the inlet of the observation chamber and the channel system, as well as between the outlet of the observation chamber and the channel system. The additional valve seats can be formed in the substrate.

These additional valve seats make it possible to close off the observation chamber from the channel system when the valve seats are closed with the membrane. This can be useful if, for example, a fluid exchange (this includes the possibility of exchanging a liquid for a gas or vice versa) is to be carried out in the channel system, but the cells are not (initially) to be affected by this. Another case is a possible disturbance of the fluid flow in the channel system, for example due to excessive unwanted flows or the ingress of air bubbles. In this case, the cells in the observation chamber can be protected by closing the valve seats. Overall, the two additional valve seats therefore enable efficient protection of biological samples in the observation chamber from potentially harmful external influences.

The channel system can also include a bypass so that the liquid can flow past the observation chamber through the channel system.

This arrangement with an additional bypass allows efficient fluid exchange in the channel system, whereby the observation chamber is not initially affected by this fluid exchange. The fluid can be flushed out of the channel system between the first connection and the second connection via the bypass.

The microfluidic device may further comprise a first liquid reservoir and a second liquid reservoir, wherein the first liquid reservoir is connected to the first fluid connection, and wherein the second liquid reservoir is connected to the second fluid connection. In particular, the first liquid reservoir and/or the second liquid reservoir may be plugged directly onto the respective connection. Alternatively, the first liquid reservoir and/or the second liquid reservoir may be formed in the substrate, in particular as a cavity in the substrate.

The liquid reservoirs are used to provide a fluid for microbiological investigations. In particular, the fluid can be conveyed from the first liquid reservoir through the channel system into the second liquid reservoir or in the opposite direction.

By attaching the liquid reservoirs directly to the connections, there is no need for any hose connections, which means that the complexity of the entire setup can be significantly reduced.

The fluid connections can be formed conically. In particular, the connections can comply with the Luer standard. The fluidic connections of the microfluidic device can have a male or female Luer or Luerlock adapter. In intended use, a tube or a liquid reservoir is plugged onto the respective connection. By using conical connections, in particular connections that comply with the Luer standard, the connections can be connected in a liquid-tight manner during filling and filling can therefore be carried out easily and reliably. With the Luer standard, the microfluidic device is compatible with a wide range of tubes and reservoirs intended for connection. Furthermore, direct attachment of liquid reservoirs to female Luer or Luerlock adapters is particularly easy.

Alternatively, the fluidic connections can be formed as barbed tee connections. It is also conceivable that some fluidic connections of the microfluidic device are conical, while others are configured as barbed tee connections.

Further, the microfluidic device may comprise a third fluidic connection and a fourth fluidic connection, and a third and a fourth liquid reservoir connected to the third and fourth fluidic connections, respectively, wherein the third and fourth connections are fluidly connected to the channel system.

This arrangement with four liquid reservoirs increases the flexibility of the microfluidic device because a plurality of liquids can be introduced into the channel system in a controlled manner by switching the corresponding valves (or valve seats). This allows the study of cells in the observation chamber under the influence of several (different) liquids.

In this case, too, all liquid reservoirs can be connected directly to the respective connection.

It is conceivable to arrange two of the described microfluidic devices in a cascade. In this case, a fluidic connection of the first microfluidic device is connected to a fluidic connection of the second microfluidic device. This principle can also be applied to a large number of microfluidic devices. In this way, the number of different liquids can be further increased and flexibility increased.

A further valve seat can be arranged between the third fluidic reservoir and the observation chamber, as well as between the fourth fluidic connection and the observation chamber. These further valve seats can also be formed in the substrate.

In this way, two further valves can be provided between the observation chamber and the third and fourth fluidic connections, so that the observation chamber can be shielded from these two connections by closing these valves. This has the advantage that it is possible to control whether or not a fluid should flow from the third or fourth liquid reservoir into the observation chamber. This allows further flexibility and more precise control of the flow in the microfluidic device.

In addition, the four valve seats can be arranged in series along the channel system, with the observation chamber arranged parallel to the series-connected valve seats.

With this arrangement, it is possible, as described above, to exchange a fluid in the channel (in particular to flush a liquid out of the channel system using gas) without affecting the cultivation conditions in the observation chamber. For this purpose, the valves formed by the valve seats between the observation chamber and the channel system, i.e. between the inlet and the channel system, and between the outlet and the channel system, are switched in such a way that the fluid does not flow through the observation chamber. Meanwhile, all other valves are in the open state. A fluid exchange can now be carried out by passing a gas through the channel system to remove the previous liquid from the channel system. As the observation chamber is separated from the channel system by the closed valve seats, the fluid exchange does not affect the observation chamber. Cells cultivated in the observation chamber thus do not experience any flow caused by the fluid exchange (e.g. a drying out due to the displacement of the liquid) and the cultivation conditions are not impaired.

Furthermore, the present invention provides a microfluidic system comprising a described microfluidic device and an actuator device, wherein the microfluidic device and the actuator device are non-destructively detachably connectable to each other such that the membrane is arranged between the substrate and the actuator device, wherein the actuator device is configured to exert a force on the membrane, wherein the first valve seat, the membrane and the actuator device form a first valve, wherein the second valve seat, the membrane and the actuator device form a second valve, and wherein the actuator device interacts with the microfluidic device such that the first valve and the second valve have a closed position and an open position, wherein in the closed position the membrane is pressed into the respective valve seat by the force, so that the associated valve can be brought into the closed position.

The microfluidic system comprises a microfluidic device and thus also provides its advantages with regard to the addition of fluids. Moreover, the microfluidic device and the actuator device cooperate in an intended use by forming said valves that control the flow in the microfluidic device.

The microfluidic device and the actuator device can be held together via a positive and/or frictional connection, for example via a plug-in connection. Holes can be formed in the substrate for this purpose. A surface of the actuator device can have pins that are engaged with the holes in the substrate. Alternatively, the microfluidic device and the actuator device can be clamped together using one or more clamps. In both cases, the membrane does not necessarily have to be specifically attached to the substrate. Instead, the membrane can be fixed to the substrate with the help of a sealing lip and then clamped to the actuator device. This type of attachment does not require any adhesives or chemical treatment of the substrate and/or membrane. If necessary, the membrane can also be easily replaced.

The actuator device can comprise as many sealing elements as the microfluidic device has valve seats. One, more or each of the sealing elements may be a piston or plunger, and wherein each of the valves is formed by one of the valve seats, the associated sealing element and the membrane. Further, each sealing element may be driven independently of the other sealing elements.

Each piston or plunger can be extended and retracted. In the extended state, the piston or plunger presses on the elastic membrane and pushes it into the corresponding valve seat. This closes the valve. When the piston or plunger is retracted again, the elastic membrane returns to its previous position and the valve is in the open state. Each piston or plunger of the actuator device interacts with a valve seat of the microfluidic device. The combination of a piston or plunger and an elastic membrane to realize a valve represents a simple, reversible valve design that is reliable over many switching cycles.

One side of each piston or plunger facing the membrane can be rounded. In particular, one, several or each of the valve seats can be chamfered.

The rounded surface of the piston or plunger has no sharp edges and thus reduces the risk of damage to the membrane.

The chamfered valve seat is characterized by a chamfer angle, which indicates the inclination of the valve seat relative to the second side of the substrate. 0° and 90° denote the limiting case of a flat valve seat. The chamfer angle can lie in a range between 20° and 70°, in particular between 30° and 60°. In this interval, it has been found that the valves seal well when closed and retain this property even after a large number of switching cycles. The chamfer also ensures that the rounded piston or plunger is always guided into a desired center position on the valve seat. The plunger is therefore self-positioning.

Alternatively, the side of the piston or plunger facing the membrane and/or the valve seat can be flat. In this case, the valve can also be sealed in the closed state.

The actuator device can comprise a drive unit that is configured to drive the sealing elements. The drive can be electromagnetic, hydraulic, electromechanical or pneumatic.

The pistons or plungers of the actuator device can be reliably driven over a large number of switching cycles by these forms of drive. The drive can be purely translational. The sealing elements can each have a guide, whereby the individual sealing elements are held in position and guided along a predetermined line. A drive using an electric motor is also conceivable. In this case, the drive is achieved by a superposition of translation and rotation, whereby the sealing elements are configured in the form of a screw and are guided in a guide with a thread or other type of gear.

The described microfluidic system, wherein said microfluidic system comprises liquid reservoirs, may further comprise a compressed air device configured to deliver liquid from one, more or each of the liquid reservoirs by means of positive pressure or negative pressure. In particular, the liquid reservoirs can be supplied with compressed air (including positive pressure and negative pressure) independently of each other. The compressed air can be used to pump the liquid through the microfluidic system. One advantage of pumping using compressed air is that the fluid (liquid) does not come into contact with a pump and its components, which reduces the risk of contamination of the liquid. In addition, the dead volume of liquid is reduced because the liquid can be conveyed directly from the liquid reservoir into the channel system instead of having to pass through a pump, for example.

Sterile filters can be arranged between the compressed air and the liquid, in particular at the phase boundary. These sterile filters prevent contamination of the liquid via the compressed air.

The microfluidic system can also include a control device for controlling the actuator device. The control device can be configured to control the drive unit of the sealing elements and thus control the opening and closing of the valves. The control can be individual for each sealing element, i.e. independent. The control device can control the sealing elements according to a predetermined sequence. In particular, the control device can be programmable for this purpose. Alternatively or additionally, the control device can be configured to pressurize the liquid reservoirs by means of the compressed air device. This provides further flexibility in the control of the microfluidic system. Furthermore, the control can be automated by the control unit. This results in a largely or completely autonomously functioning microfluidic system after prior programming. In particular, the supply of liquid, nutrients, etc. to the observation chamber is automated. This not only increases the simplicity of operation, but also the reproducibility of experimental results compared to a manual actuation.

The actuator device may have a smaller area than the microfluidic device such that the microfluidic device protrudes beyond the actuator device when the microfluidic device and the actuator device are connected, wherein the observation chamber is formed in the substrate of the microfluidic device, and wherein the observation chamber is located in the portion of the microfluidic device that protrudes beyond the actuator device. In other words, when the microfluidic device having an observation chamber formed in the substrate and the actuator device are connected to each other, the observation chamber does not overlap with the actuator device.

Typically, the cells that are to be examined are located in the observation chamber. Accordingly, it should be possible to perform microscopy on the cells in the observation chamber. Since the actuator device does not overlap with the observation chamber, an objective can instead be arranged in close proximity to the observation chamber to allow microscopy with high resolution.

The microfluidic device may be disposable for single use. The actuator device can be reusable.

The fluid is only in contact with the microfluidic device, but not with the actuator device. This means that parts that come into contact with the fluid are designed for single use. After use, the microfluidic device can simply be disposed of and does not need to be cleaned. In addition, the sterility of the components that come into contact with the fluid can easily be maintained by using a new microfluidic device for a new application. If the microfluidic device is manufactured by injection molding, it can be produced inexpensively and in large quantities. On the other hand, the actuator device with the sealing elements etc. can be used several times. It does not come into contact with the fluid and therefore does not need to be cleaned after each use, nor does it cause potential contamination of the fluid.

The actuator device can be used with a variety of different microfluidic devices, making it a universal actuator device. For this purpose, the actuator device comprises a plurality of sealing elements at predefined positions. The microfluidic device is adapted to this actuator device in that the valve seats are also provided at predefined positions in the substrate and interact with the sealing elements. The universal actuator device comprises more or the same number of sealing elements as the microfluidic device comprises valve seats. In the event that there are fewer valve seats than sealing elements, those sealing elements that do not interact with any valve seat are not controlled in the actuator device. This arrangement therefore allows an actuator device to be used with a large number of different microfluidic devices. This is particularly useful because the actuator device is a reusable article and is intended for a plurality of cycles of use and/or is intended to interact with a plurality of microfluidic devices.

In the following and in the figures, the same reference signs are used for the same or corresponding elements in the various embodiments, unless otherwise indicated.

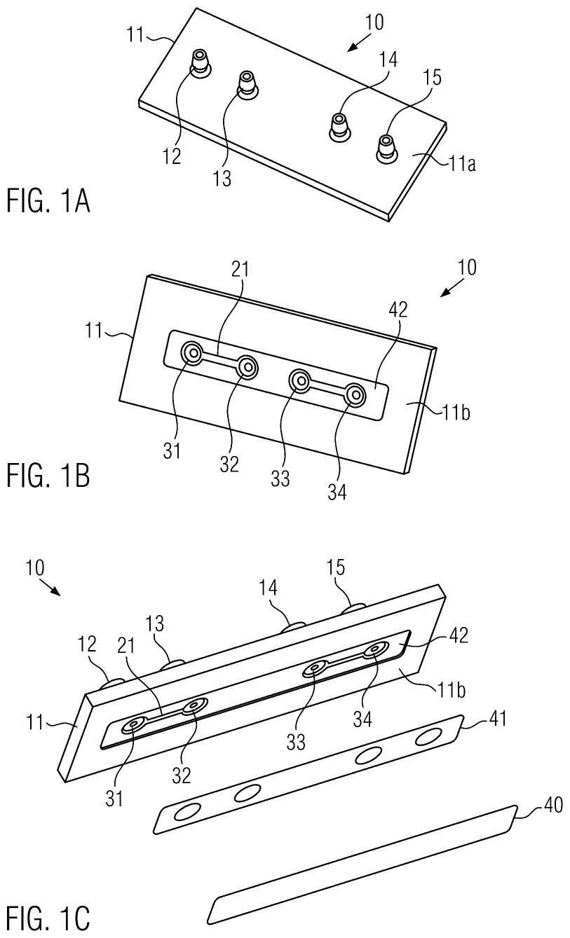

Referring now to the figures, FIGS. 1A to 1C show different views of a microfluidic device 10 according to the invention, which initially comprises a substrate 11, a first fluidic connection 12, a second fluidic connection 13, a third fluidic connection 14, a fourth fluidic connection 15 and a trench 21.

The microfluidic device 10 is suitable for use with liquids and gases.

FIG. 1A shows a schematic oblique view of the microfluidic device 10, with a first side 11a of the substrate 11 facing upwards. The four fluidic connections 12, 13, 14, 15 are also arranged on this first side 11a. The connections 12, 13, 14, 15 may be attached to the substrate 11, but they may also be an integral part of the substrate 11. In the latter case, the connections 12, 13, 14, 15 and the substrate 11 are made from the same material and in particular from a single piece. This has the advantage of greater stability and the possibility of simplified manufacture, above all because fewer handling steps are required to assemble the microfluidic device 10. For example, it is possible to produce the substrate 11 including the connections 12, 13, 14, 15 by injection molding in one manufacturing step.

The substrate 11 can be transparent in the visible wavelength range. In some applications, however, it is also sufficient if the substrate 11 is merely translucent or even opaque, in particular if no optical examinations need to be carried out on the substrate 11.

FIG. 1B shows a schematic oblique view of the microfluidic device 10, with a second side 11b of the substrate 11 facing upwards, the second side 11b being opposite the first side 11a. For simplification, no membrane is shown in this illustration, but only in FIG. 1C. In this second side 11b, there is a recess in the surface of the substrate 11. This recess serves to accommodate a membrane. There is also a trench 21 in the second side 11b. By covering it with a film (described below), this trench 21 becomes a channel system. Where there is a connection on the first side 11a, there is a valve seat on the second side 11b. A liquid that is introduced at one of the connections flows through the substrate 11 to the second side and passes through the valve seat. The valve seats 31, 32, 33, 34 are thus located opposite the four connections 12, 13, 14 and 15 on the second side 11b.

The trench 21 formed on the second side 11b of the substrate 11 connects a pair of valve seats to each other, so that the fluid can flow from one valve seat through the channel system to another valve seat.

The structure of the channel system or trench 21 may be configured individually for each microfluidic device, and further examples are explained with reference to the following embodiments.

FIG. 1C is an exploded view of the microfluidic device 10. The microfluidic device 10 comprises the previously explained substrate 11 (with the connections and valve seats) and an elastic membrane 40. The membrane 40 is attached to the substrate 11 by means of a double-sided adhesive film 41. A recess 42 is configured in the second side 11b of the substrate 11, the shape of which corresponds to the shape of the membrane 40 and the adhesive film 41. The adhesive film 41 is placed in the recess 42. The adhesive film 41 leaves out the valve seats 31, 32, 33, 34, which is why the circular holes are provided in the adhesive film 41 (the holes are adapted to the shape of the valve seats. Since the valve seats are round in the case shown, this also applies to the holes in the adhesive film 41). The membrane 40 is placed on the adhesive film 41 fixed in the recess 42 and adhered thereto. In this respect, the trench 21 is covered by the adhesive film 41 and the channel system 20 is formed.

The individual parts of this microfluidic device 10, i.e. the substrate 11, the adhesive film 41 and the membrane 40, are easy to manufacture and the assembly of the microfluidic device 10 is also uncomplicated.

Alternatively, the membrane 40 can also be placed in the recess without the adhesive film 41. In this case, the membrane is clamped into the recess 42.

In addition to the valve seats, the adhesive foil 41 can also leave out the trench 21. In this case, the channel system is formed by covering the trench 21 with the membrane 40. As a result, cells that are passed through the channel system 20 cannot adhere to the adhesive film 41. Similarly, substances from the surface of the adhesive film (e.g. the sticky film) are not released into the fluid flow in the channel system, which avoids possible unwanted contamination.

In particular, the recess 42 has the same thickness/depth as the adhesive film 41 and the membrane 40 in total. As a result, the underside of the microfluidic device is flat when the microfluidic device 10 has been assembled. This has the advantage that a flat surface is available for microscopic examinations, which has a positive influence on the optical examinations. If no adhesive film is used, the depth of the recess 42 corresponds to the thickness of the membrane 40.

The membrane 40 may be a silicone membrane and in particular comprise or consist of Elastosil. Similarly, the membrane may comprise or consist of a thermoplastic elastomer such as Flexdym. Alternatively, the membrane may comprise or consist of a COC. The thickness of the membrane 40 is in the range between 5 μm and 5 mm. Thus, the membrane has a high longevity over a large number of switching cycles and at the same time only a small force is required to press the membrane 40 into the associated valve seat.

This microfluidic device 10 allows fluids to be added to the channel system in a targeted manner via its fluidic connections. The addition is controlled by the associated valve seat, whereby the valve seat can be closed by the membrane if necessary and the addition of fluid can be blocked. A liquid that is already in the channel system is mixed by the addition of further liquids, but an existing flow is not obstructed. This is particularly advantageous if this flow is used for cultivating microbiological samples, as undesirable interference with the cultivation conditions can be avoided with the aid of this microfluidic device 10. Furthermore, the present microfluidic device has a simple and compact design. In other words, the microfluidic device enables efficient and precise control of fluid flow in a channel system while maintaining a compact and simple design.

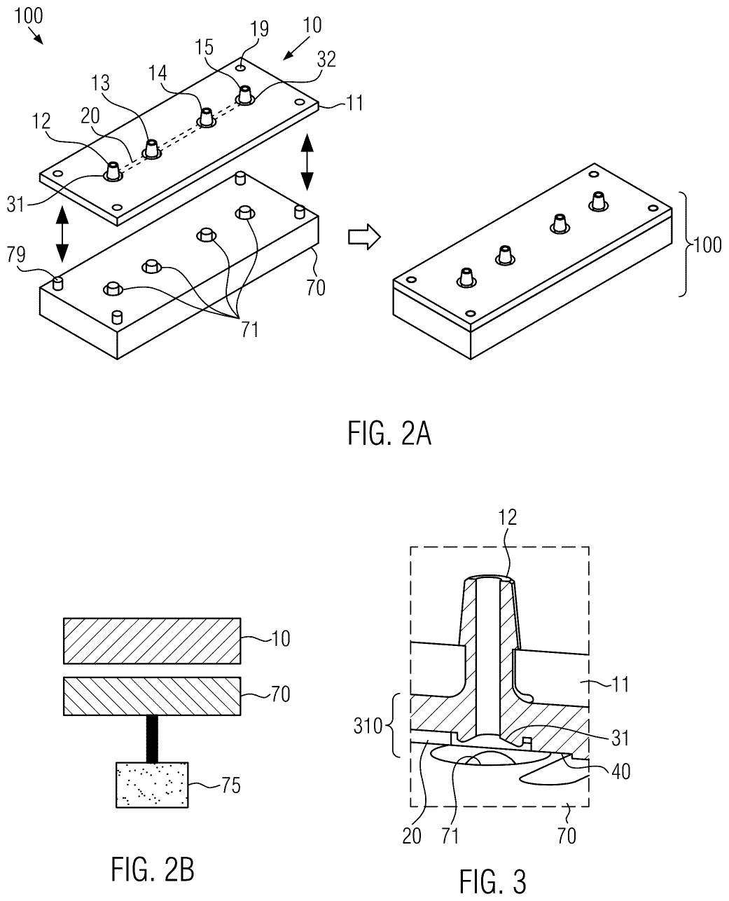

FIG. 2A shows an oblique view of a microfluidic system 100 comprising a microfluidic device 10 and an actuator device 70. The microfluidic device 10 may be configured as illustrated in FIG. 1A-C.

The actuator device 70 comprises a plurality of sealing elements 71, which in the present case are formed as pistons or plungers. The sealing element 71 can be driven electromechanically, electromagnetically, hydraulically or pneumatically, for example by a compressed air valve. Optionally, the actuator is bistable and has a locking mechanism. The sealing elements 71 can be extended and retracted, whereby the sealing elements 71 are shown in the figure in the extended state. The drive elements 71 are driven by a corresponding drive unit 75 (see FIG. 2B), which can be a servomotor, an electric motor, a solenoid or a pneumatic or hydraulic cylinder, for example. A bistable actuator assumes various stable switching states in a de-energized state. The stable states can be realized by a mechanical interlock, magnetic remanence or a permanent magnet. In the case of the solenoid, the stable states are generated via a permanent magnet and a return spring.

The block diagram in FIG. 2B shows this structure of a microfluidic system 100 with a microfluidic device 10, an actuator device 70 and a drive unit 75.

The microfluidic device 10 and the actuator device 70 can be connected to each other as shown in FIG. 2A, whereby the connection can be released non-destructively. For example, the microfluidic device 10 and the actuator device 70 can be plugged together and securely, but also detachably, connected by a positive fit. Alternatively, the two devices can be clamped together using additional clamps (not shown). In the figure, the actuator device 70 has a plurality of pins 79. At the same time, the substrate 11 has a plurality of holes 19. When assembling the microfluidic system 100, the pins 79 are inserted into the holes 19, thus creating the aforementioned form-fit, yet non-destructively detachable connection (plug-in connection) between the microfluidic device 10 and the actuator device 70.

When the microfluidic device 10 and the actuator device 70 are connected, the membrane 40 is arranged between the substrate 11 and the actuator device 70. Further, the microfluidic device 10 and the actuator device 70 are adapted to each other such that each of the valve seats 31, 32, 33, 34 overlaps with one of the sealing elements. A sealing element 71, the membrane 40 and a valve seat 31, 32, 33, 34 form a valve in this configuration, each of the actuator elements 71 being associated with a valve seat 31, 32, 33, 34. The function of a valve formed in this way is explained in more detail with reference to FIG. 3.

The microfluidic device 10 and the actuator device 70 may have the same dimensions of their cross-section. For example, the microfluidic device may have dimensions of 35 cm by 35 cm, in particular 15 cm by 20 cm, further in particular 5 cm by 5 cm. Similarly, the dimensions may conform to an ANSI/SLAS standard for multiwell plates. The actuator device 70 can have the same length and width as the microfluidic device 10.

FIG. 3 shows a detailed view of a valve 310 as it appears in a microfluidic system 100. The microfluidic system 100 comprises a microfluidic device 10 and an actuator device 70, which are connectable to each other. In the case shown, which describes an intended use of the microfluidic system 100, the two components are connected.

The microfluidic device 10 comprises a substrate 11, a connection 12 on the first side 11a of the substrate 11, and a valve seat 31 on the second side 11b of the substrate 11, the second side 11b being opposite the first side 11a. A through opening leads through the substrate 11 from the connection 12 to the valve seat 31. The valve seat 31 itself is chamfered, i.e. funnel-shaped.

The actuator device 70 comprises a sealing element 71 in the form of a plunger. The plunger has a rounded surface that faces the membrane 40. In the figure, the sealing element 71 is shown in a retracted state, in which the plunger is recessed in the actuator device 70. The sealing element 71 can be brought into an extended state, for example, by an electromechanical, electromagnetic or hydraulic drive. The plunger is always automatically guided into a central position by the chamfered valve seat in order to optimally seal the valve seat. The valve seat is therefore self-positioning for the plunger.

The valve seat 31 can also be rounded and interact with a likewise rounded surface of the sealing element 71.

However, the invention is not limited to this shape of sealing element 71 and valve seat 31. Alternatively, the valve seat can be flat. Suitably, the sealing element 71 can also be configured in the form of a plunger with a flat surface. Rounded edges on the flat plunger surface are also conceivable in order to avoid possible damaging of the membrane from sharp edges.

The mode of operation of the valve 310 and the microfluidic system 100 is also apparent from this embodiment. In the extended state, the sealing element 71 exerts a force on the membrane 40 by pressure and presses it into the valve seat 31, whereby the valve is in the closed state. The chamfered valve seat 31 in combination with the rounded surface of the sealing element contributes to a better seal in the closed state of the valve 310. Consequently, when the valve 310 is closed, no fluid can get from the connection 12 into the channel system 20 or vice versa. As soon as the sealing element 71 is brought into the retracted state, the membrane 40 returns to its previous position due to its elasticity and the valve 310 is brought into the open state. A fluid can now flow from the connection 12 into the channel system 20 or from the channel system 20 to the connection 12.

The channel system 20 is further designed such that a channel is located below the valve seat 31, but widens there so that it is not closed by the closing of the valve 310. This means that a fluid can flow around the valve seat 31 while remaining in the channel system 20 when the valve 310 is closed. When the valve 310 is open, the fluid can enter the channel system 20 from the associated fluidic connection through the valve seat 31. This means that a closed valve 310 need not cause the flow to stop, but the fluid can continue to remain in the channel system 20 without flowing through the valve seat 31.

Liquid reservoirs can be connected to one or more of the fluidic connections, so that when the valves are open, the liquid flows from the respective liquid reservoir into the channel system. The targeted opening and closing of the valves controls which liquids flow into the channel system. By controlling the opening time, the amount of the respective liquid that is introduced into the channel system can also be controlled. This makes it possible to create a precise mixing ratio of liquids in the channel system. Furthermore, the channel system can be connected to an observation chamber in which a microbiological sample is located. This sample is supplied with the liquid mixture from the channel system. If the liquids are nutrient solutions, for example, the sample can be precisely supplied with nutrients. In this respect, the microfluidic system according to FIG. 2 can be used to provide a precisely adjusted nutrient solution for microbiological samples.

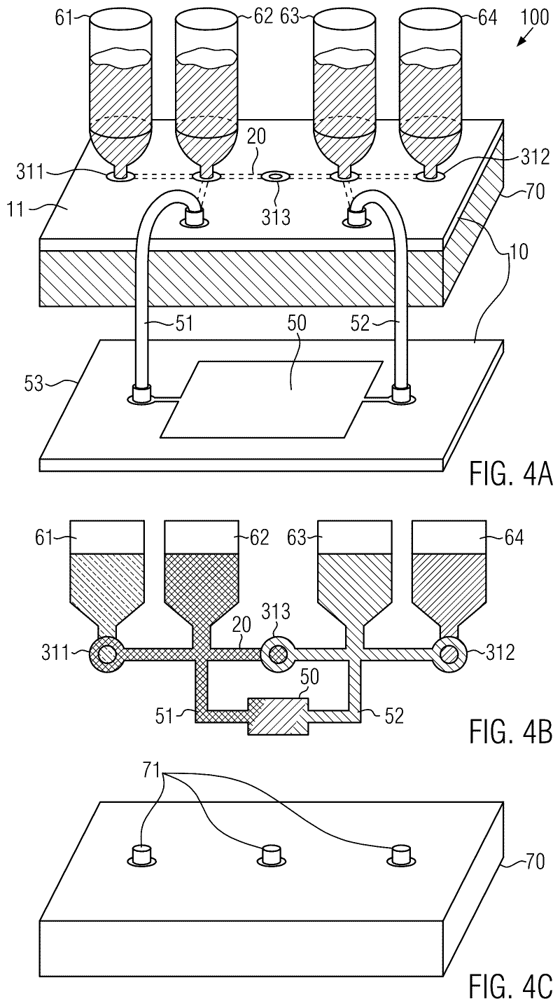

Another embodiment of the microfluidic system 100 is shown in FIG. 4. This is essentially based on the first embodiment (FIG. 2A), thus comprising a microfluidic device 10 and an actuator device 70. The microfluidic device 10 comprises a substrate 11, wherein a channel system 20 and three valve seats are configured in the substrate 11, as well as a membrane. The actuator device 70 comprises three sealing elements 71 that interact with the three valve seats when the microfluidic device 10 and the actuator device 70 are connected together.

The microfluidic system 100 has four liquid reservoirs 61, . . . , 64, which are plugged directly onto the connections of the microfluidic device 10. Valves 311 and 312 are located between the first liquid reservoir 61 and the channel system 20, and between the fourth liquid reservoir 64 and the channel system, so that fluid from the respective reservoir can only flow into the channel system 20 when the respective valve is open. Fluid from the second liquid reservoir 62 and the third liquid reservoir 63, on the other hand, can enter the channel system 20 directly, as no valve is arranged here. The valve 311 is formed by the valve seat 31, one of the drive elements 71 and the membrane. The valve 312 is formed by the valve seat 32, one of the drive elements 71 and the membrane.

By attaching the liquid reservoirs 61-64 directly to the connections, there is no need for any hose connections, so that the complexity of the entire structure can be reduced. This is accompanied by simplified sterile maintenance of the device, as fewer potentially contaminable components are involved. Another advantage is that the dead volume is reduced because no hoses need to be filled with the liquid. Thus, a small amount of liquid is sufficient, which is delivered directly from the respective liquid reservoir 61-64 into the channel system 20. The elimination of hose connections also has the advantage that any temperature gradients along the transport path of the fluid do not occur. In the case of liquids, this reduces the possibility of bubble formation. Air bubbles could have negative effects on the cultivation of the cells or falsify test results.

An observation chamber 50 is connected to the channel system 20 via connecting hoses, with the hose 51 serving as the inlet and the hose 52 as the outlet of the observation chamber 50 (the reverse assignment is also possible, whereby the assignment basically depends on the direction of flow). The observation chamber 50 is configured in a separate substrate 53 and is considered to be part of the microfluidic device 10.

The liquid reservoirs 62, 63 may contain, for example, a nutrient solution that is delivered through the observation chamber 50. The observation chamber 50 may contain cells or cell aggregates that are to be cultivated under controlled environmental conditions. By supplying the nutrient solution, the cells in the observation chamber 50 are supplied with nutrients. When the valve 311 is opened, a further liquid can be introduced from the first liquid reservoir 61 into the channel system 20, where it mixes with the nutrient solution. The dosage of the further liquid can be adjusted flexibly and in a controlled manner by the duration of the opening and/or a pressurization of the first liquid reservoir 61. The same applies to a further liquid in the fourth liquid reservoir 64. In this way, further liquids can be introduced into the channel system 20 in a simple and controlled manner, where they mix with the nutrient solution and are supplied to the cells in the observation chamber 50.

In the substrate 11 there is a further valve seat 33 in fluidic connection with the channel system 20, which is arranged parallel to the connections for the observation chamber 50. This valve seat 33 is thus arranged in a bypass, which allows a fluid to be conveyed through the channel system 20 past the observation chamber 50. This bypass is part of the channel system and can also be referred to as a bypass channel. Together with the actuator device 70 and the observation chamber 50, a valve 313 is thus formed which is arranged parallel to the observation chamber 50. This valve 313 can be referred to as a bypass valve. The bypass valve 313 can be used to set whether the fluid is to flow exclusively through the observation chamber between the third connection 14 and the fourth connection 15, or whether a portion of the fluid is to flow via the bypass. For example, the flow in the observation chamber 50 can be reduced by opening the bypass valve 313.

A schematic circuit diagram of this microfluidic system is shown in FIG. 4B. The two liquid reservoirs 61, 64 are isolated from the channel system by the respective valve 311, 312. The other two liquid reservoirs 62, 63, on the other hand, are connected directly to the channel system without valves. As a result, the liquid from the liquid reservoirs 62, 63 can also be supplied directly to the observation chamber 50. Liquid from the liquid reservoirs 61, 64 is supplied to the channel system 20 by opening the respective valve. The function of the bypass valve 313 is also shown in more detail. When the bypass valve 313 is closed, the fluid on the path between the liquid reservoirs 62, 63 flows exclusively through the observation chamber 50. By opening the bypass valve 313, on the other hand, part of the fluid can also flow via the bypass and the flow through the observation chamber 50 is reduced.

To further illustrate the valve arrangement and its function, the actuator device 70 is shown separately from the microfluidic device 10 in FIG. 4C. The actuator device 70 comprises three sealing elements 71 which interact with the three valve seats of the microfluidic device 10. A cross-sectional area of the substrate 11 corresponds in particular to a cross-sectional area of the actuator device 70. The microfluidic device 10 and the actuator device 70 can be plugged together with a plug-in connection, as explained with respect to FIG. 2.

This configuration can be used in particular if the fluid to be conveyed through the channel system 20 and the observation chamber 50 contains certain cell types such as blood cells or immune cells. In particular, these cells should not be thermally or mechanically stressed because the cells could otherwise be activated, which is generally undesirable. Such (especially mechanical) stress occurs in particular at a valve, because a slightly turbulent flow occurs at such a narrow point and the cells are subjected to high stress through contact with the substrate, the membrane or each other. This stress can be avoided in this embodiment. Typically, the cells are conveyed through the observation chamber between the third liquid reservoir 63 and the fourth liquid reservoir 64. This section is free of valves, so that the described stress does not occur or occurs to a reduced extent.

In principle, this embodiment of the microfluidic system can be extended by providing a cascade instead of a single microfluidic device and actuator device. In this case, a further microfluidic device is arranged in the microfluidic system described instead of the observation chamber and the two tubes 51, 52 are connected to the further microfluidic device, where the tubes take the place of the liquid reservoirs 61, 64. The further actuator device is connected to the microfluid device as described above and the observation chamber can be connected to the second microfluid device. Of course, this principle can be extended accordingly to more than two microfluidic devices and actuator devices. This has the advantage that more (different) liquids can be fed into the system and a more flexible design is possible. For example, two microfluidic devices connected in series allow the addition of up to six liquids, while one such microfluidic device allows a maximum of four liquids.

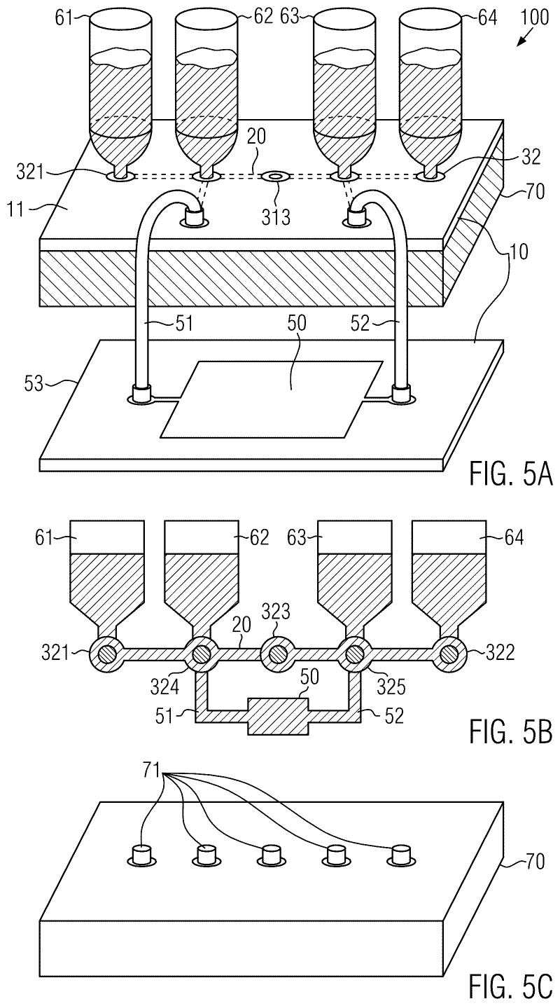

FIG. 5A shows another embodiment of the microfluidic system 100, which differs from the second embodiment by two additional valves 324 and 325 arranged between the third connection 14 and the observation chamber 50, and between the fourth connection 15 and the observation chamber 50. Moreover, this embodiment also comprises a microfluidic device 10 having a substrate 11, liquid reservoirs 61, 62, 63, 64, a channel system 20, an observation chamber 50, and an actuator device 70.

Between the second liquid reservoir 62 and the channel system 20, as well as between the third liquid reservoir 63 and the channel system 20, there is a valve 324 and 325 respectively, which are each formed by the interaction of a valve seat, the membrane and a sealing element of the actuator device. Thus, by opening and closing these valves, a flow between the second liquid reservoir 62 and the channel system 20, as well as between the third liquid reservoir 63 and the channel system 20, can be controlled (which is not possible in the embodiment according to FIG. 4). For clarification, the shape of the actuator device 70 is also shown in FIG. 5C. This comprises a total of five sealing elements 71 which, together with the corresponding five valve seats, form valves 321, 322, 323, 324 and 325.

As can be seen from the arrangement shown, the valves 321 . . . 325 are arranged in series with one another and parallel to the observation chamber 50. This arrangement can be used to isolate the observation chamber 50 from the channel system 20 by closing the valves 324 and 325, while the fluid is flushed from the channel system 20.

Reference is made to the schematic diagram of the valve arrangement in FIG. 5B to illustrate this relationship. As shown therein, an opening of the bypass valve 323 allows a direct connection between the second liquid reservoir 62 and the third liquid reservoir. If air or another gas (e.g. argon) is now introduced at one of the liquid reservoirs 62, 63, a liquid is removed from the channel system 20. At the same time, this process has no influence on the observation chamber 50 because it can be fluidically separated from the channel system 20 by closing the valves 324 and 325. A new liquid can then be introduced via the liquid reservoirs 62, 63. This change of medium takes place in particular without unnecessary waste of the new fluid, because the old fluid has been practically completely displaced by air and the new fluid can therefore be used directly.

Flushing a liquid out of the channel system using a gas (e.g. air, argon, etc.) is particularly effective. As a result, the liquid is removed from the channel system with virtually no residue and using a small volume of gas, after which a new liquid can be introduced.

In principle, the microfluidic systems of FIGS. 4 and 5 can also comprise a universal actuator device configured for use with both associated microfluidic devices. This universal actuator device comprises, for example, five sealing elements that can interact with the five valve seats 31-35 of the embodiment shown in FIG. 5. The embodiment according to FIG. 4 comprises only three valve seats (valves) 31, 32, 33, which are located at the same position as the associated valve seats in the embodiment according to FIG. 5. Therefore, the actuator device of the embodiment according to FIG. 5 can also be used for the embodiment according to FIG. 4 if only those sealing elements are used which interact with the three valve seats 31, 32, 33.

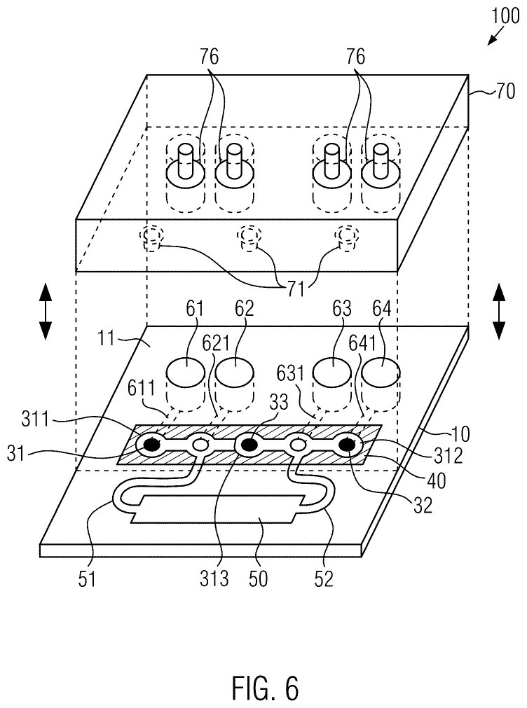

The microfluidic system 100 according to a further embodiment in FIG. 6 is essentially based on the embodiment according to FIG. 5 and comprises the corresponding valve arrangement or arrangement of valve seats in the substrate. This is therefore not described again in detail.

First of all, the microfluidic system 100 comprises an observation chamber 50 integrated in the substrate 11 of the microfluidic device 10, which is in the form of a cavity in the substrate 11 and is connected to the channel system 20 via an inlet 51 and an outlet 52. In this case, the inlet 51 and outlet 52 are themselves in the form of a channel in the substrate 11. Alternatively, the observation chamber 50, like the channel system 20, may be configured in the form of a trench in the surface of the substrate 11. This trench is fluidically sealed by the membrane 40, adhesive film or another element, resulting in the observation chamber 50.

Further, the four liquid reservoirs 61-64 are formed as cavities in the substrate 11 of the microfluidic device 10. The individual liquid reservoirs are connected to the valve arrangement and/or the channel system 20 via associated channels 611, 621, 631, 641. The channel 611 connects the liquid reservoir 61 to the valve seat 31, the channel 621 connects the liquid reservoir 62 to the channel system 20, the channel 631 connects the liquid reservoir 63 to the channel system 20 and the channel 641 connects the liquid reservoir 64 to the valve seat 32. The valve seat 33 forms part of a bypass valve as already described in FIG. 4A. Overall, the microfluidic system 100 described herein realizes the connections of the liquid reservoirs, the valve seats, the channel system and the observation chamber shown in FIG. 5B.

The actuator device 70 comprises three sealing elements 71 that cooperate with the valve seats 31, 32, 33 to form the valves 311, 312, 313. In addition, the actuator device 70 comprises a total of four pump units 76. These are shown as pistons in the embodiment shown, but are not limited to this form. The pump units 76 are configured to pressurize the four liquid reservoirs 61-64 and thus deliver the liquid. Furthermore, the pump units can have the function of venting the respective reservoirs. The actuator device 70 is connected to the microfluidic device 10 and thus forms the microfluidic system 100. A plug-in connection or a clamp connection, for example, can be considered as connection types. A further difference to the embodiment example according to FIG. 5A-C is that the microfluidic device 10 and the actuator device 70 do not have the same dimensions in length and width (i.e. their cross-sectional area), so that the observation chamber 50 and the actuator device 70 do not overlap with each other when the microfluidic system 100 is assembled. In other words, the microfluidic device 10 extends beyond the actuator device 70. This has the advantage that it should be possible to perform microscopy on the observation chamber 50 by providing sufficient optical access. Typically, there are cells, cell aggregates or the like in the observation chamber which are to be examined microscopically. For this purpose, an objective lens (not shown) must be brought as close as possible to the cells from the first side or the second side of the substrate 11. The configuration described is a simple way of creating the necessary free space for the lens.

For the purpose of said microscopic examinations, the substrate 11 can be transparent in the area of the observation chamber 50, in particular in the visible wavelength range. If the observation chamber 50 is sealed by the membrane 40, this requirement also applies to the membrane 40. In the other areas of the microfluidic device, no high optical requirements apply to the substrate 11. Here, the substrate 11 can therefore also be merely translucent or even opaque.

This design does not require any liquid tubing because all the necessary connections between the elements (in particular liquid reservoirs, channel system and observation chamber) are present in the substrate in the form of channels. This considerably reduces the complexity of the structure. In addition, the risk of contamination and bubble formation is also reduced by reducing the number of tube connections.

It is understood that an observation chamber integrated in the substrate and/or the liquid reservoirs integrated in the substrate can also be combined with the embodiment example according to FIG. 5. The observation chamber can therefore also be integrated in the substrate if a valve or valve seat is provided between the second liquid reservoir and the observation chamber, as well as between the third liquid reservoir and the observation chamber. In this case, too, the actuator device and the observation chamber should not overlap so that microscopy of cells in the observation chamber is possible. A microfluidic device as in FIG. 1, in which the observation chamber is formed in the substrate, is also conceivable. If necessary, the actuator device can also comprise the pump units described.

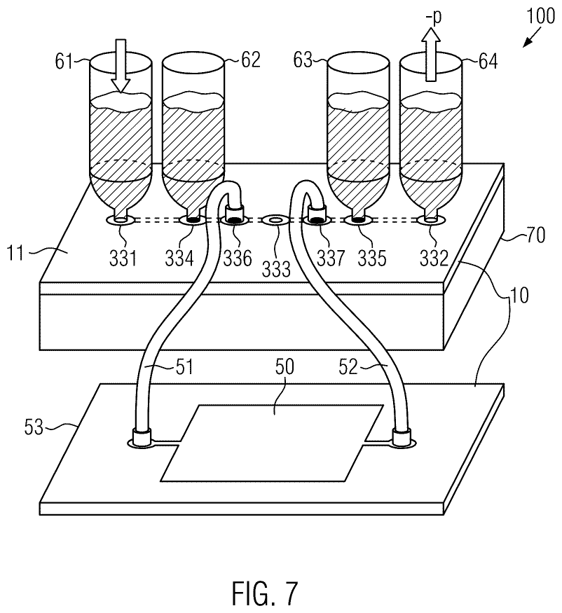

FIG. 7 shows a further embodiment of the microfluidic system and a possible application example. Like the embodiment example according to FIG. 5A, the microfluidic system 100 comprises four liquid reservoirs 61, 62, 63, 64, which are plugged directly onto fluidic connections of the microfluidic device 10. Here, the liquid reservoirs 61, 62, 63 are open to the atmosphere and the liquid reservoir 64 is connected to a pump (not shown) which can generate a negative pressure-p.

A total of seven valve seats are configured in the substrate 11 of the microfluidic device, which together with the associated sealing elements of the actuator device 70 form the valves 331-337. The microfluidic device further comprises an observation chamber 50 as already described in the embodiment example of FIG. 4A. Further explanations are therefore dispensed with at this point.

With this arrangement, it is possible to “draw” liquid from the reservoir 61 through the channel system 20 into the liquid reservoir. To do this, the seven valves are brought into the configuration shown, with a black full circle indicating a closed valve and a white full circle indicating an open valve. Valves 331, 332 and 333 are therefore open, while valves 334, 335, 336 and 337 are closed. As a result of the negative pressure applied to the liquid reservoir 64, the liquid flows from the liquid reservoir 61 through the channel system 20, past the observation chamber 50, via the bypass valve 333 to the liquid reservoir 64. Consequently, pressure only needs to be applied to one liquid reservoir. The direction of flow in the channel system 20 is determined solely by the position of the valves (open or closed)

This variant demonstrates the aforementioned advantage that the microfluidic system can manage with a minimum of hose connections and at the same time offers a wide range of possible uses. As a result, the microfluidic system described here is much simpler than existing solutions, reduces unfavorable dead volumes and reduces the likelihood of unwanted air bubbles forming.

A common principle of the microfluidic system described, in particular of the embodiments according to FIGS. 4 to 7, is the so-called “Common Rail Principle” (CRP). Here, the observation chamber is separated from the channel system by two valves (between the channel system and the inflow, and between the channel system and the outflow). The channel system is connected to a number of connections and associated liquid reservoirs.

The CRP offers a number of advantages. For example, the microfluidic system can be prepared by filling the channel system with a specific fluid using small volumes. Experiments on biological samples can begin as soon as the fluid enters the observation chamber. Since the liquid reservoirs can be plugged directly onto the connections of the microfluidic device, only a very small dead volume is created and a smaller amount of fluid is actually required. Another aspect, as previously described, is that the channel system can be flushed without affecting the observation chamber. This is particularly efficient when a liquid is flushed out of the channel using air, because the liquid is removed by flushing with air with virtually no residue.

1. A microfluidic device comprising:
a substrate,
a channel system arranged in the substrate,
fluidic connections comprising a first fluidic connection and a second fluidic connection for supplying a fluid to the channel system, the fluidic connections being arranged on a first side of the substrate,
valve seats comprising a first valve seat and a second valve seat, both formed on a second side of the substrate that is opposite to the first side, and
an elastic membrane covering at least part of the second side of the substrate, including the valve seats,
wherein the first valve seat is arranged in such a way that the fluid can flow from the first connection through the first valve seat into the channel system, and
wherein the second valve seat is arranged such that the fluid can flow from the second connection through the second valve seat into the channel system.

2. The microfluidic device according to claim 1, wherein the channel system is at least partially formed in the form of a trench on the second side of the substrate, and wherein the membrane fluidically seals the trench.

3. The microfluidic device according to claim 1, wherein at least one of:
the membrane is a silicone membrane, in particular comprising Elastosil, or
the membrane has a thickness in a range between 5 μm and 5 mm.

4. The microfluidic device according to claim 1, wherein a double-sided adhesive film is further arranged between the substrate and the membrane, wherein the double-sided adhesive film leaves out at least the valve seats, and wherein the membrane is attached to the substrate by means of the double-sided adhesive film.

5. The microfluidic device according to claim 1, further comprising an observation chamber that is fluidically connected to the channel system via an inlet and an outlet.

6. The microfluidic device according to claim 5, wherein a further valve seat is arranged each between the inlet of the observation chamber and the channel system, and between the outlet of the observation chamber and the channel system.

7. The microfluidic device according to claim 1, further comprising a first liquid reservoir and a second liquid reservoir, wherein the first liquid reservoir is connected to the first fluidic connection, and wherein the second liquid reservoir is connected to the second fluidic connection.

8. The microfluidic device according to claim 7, further comprising:
a third fluidic connection and a fourth fluidic connection, and
a third liquid reservoir and a fourth liquid reservoir connected to the third fluidic connection and the fourth fluidic connection, respectively,
wherein the third fluidic connection and the fourth fluidic connection are fluidically connected to the channel system.

9. The microfluidic device according to claim 8, wherein a valve seat is arranged between the third fluidic connection and the observation chamber and between the fourth fluidic connection and the observation chamber.

10. A microfluidic system, comprising:
a microfluidic device comprising:
a substrate,
a channel system arranged in the substrate,
fluidic connections comprising a first fluidic connection and a second fluidic connection for supplying a fluid to the channel system, the fluidic connections being arranged on a first side of the substrate,
valve seats comprising a first valve seat and a second valve seat, both formed on a second side of the substrate that is opposite to the first side, and
an elastic membrane covering at least part of the second side of the substrate, including the valve seats,
wherein the first valve seat is arranged in such a way that the fluid can flow from the first connection through the first valve seat into the channel system, and
wherein the second valve seat is arranged such that the fluid can flow from the second connection through the second valve seat into the channel system, and an actuator device,
wherein the microfluidic device and the actuator device are non-destructively detachably connectable to one another in such a way that the elastic membrane is arranged between the substrate and the actuator device,
wherein the actuator device is configured to exert a force on the elastic membrane,
wherein the first valve seat, the elastic membrane and the actuator device form a first valve,
wherein the second valve seat, the elastic membrane and the actuator device form a second valve, and
wherein the actuator device interacts with the microfluidic device such that the first valve and the second valve each have a closed position and an open position, wherein in the closed position the elastic membrane is pressed by the force into a respective one of the first valve seat or the second valve seat so that the associated valve can be brought into the closed position.

11. The microfluidic system according to claim 10, wherein the actuator device comprises as many sealing elements as the microfluidic device has valve seats, wherein each of the sealing elements is a piston or plunger, and wherein each of the first valve and the second valve is formed by one of the valve seats, the associated sealing element and the membrane.

12. The microfluidic system according to claim 11, wherein a side of each piston or plunger facing the elastic membrane is rounded.

13. The microfluidic system according to claim 11, wherein the actuator device comprises a drive unit configured to drive the sealing elements electromagnetically, hydraulically, electromechanically and/or pneumatically.

14. The microfluidic system according to claim 10,
wherein the microfluidic device has a larger area than the actuator device such that the microfluidic device extends beyond the actuator device when the microfluidic device and the actuator device are connected to each other,
wherein an observation chamber is formed in the substrate of the microfluidic device, and
wherein the observation chamber is located in the part of the microfluidic device that extends beyond the actuator device.

15. The microfluidic system according to claim 10, wherein at least one of:
the microfluidic device is a disposable single-use device, or
the actuator device is reusable.

16. The microfluidic device according to claim 5, wherein the observation chamber is formed in the substrate.

17. The microfluidic device according to claim 6, wherein the channel system comprises a bypass, so that the fluid can flow through the channel system past the observation chamber.

18. The microfluidic device according to claim 7, wherein at least one of the first liquid reservoir or the second liquid reservoir is placed directly onto the respective connection.

19. The microfluidic device according to claim 9, wherein the valve seats are arranged in series along the channel system, and wherein the observation chamber is arranged parallel to the valve seats connected in series.

20. The microfluidic system according to claim 12, wherein each of the valve seats is chamfered.
